(19) United States
(12) Patent Application Publication
(10) Pub. No.: US 2026/0014326 A1
(43) Pub. Date: Jan. 15, 2026

(54) SAFETY SYRINGE

(71) Applicant: Genray Hangzhou Life Technology Co.,Ltd., Hangzhou, Zhejiang (CN)

(72) Inventor: Chaoyu HU, Hangzhou, Zhejiang (CN)

(21) Appl. No.: 19/113,721

(22) PCT Filed: Sep. 8, 2023
(86) PCT No.: PCT/CN2023/117727

§ 371 Date: Mar. 20, 2025

(30) Foreign Application Priority Data
Sep. 21, 2022 (CN) 202222499926.2
Jul. 26, 2023 (CN) 202321983167.5

(51) Int. Cl. A61M 5/32; A61M 5/20

(52) U.S. Cl. CPC A61M 5/3232; A61M 5/2033; A61M 5/3202

(57) ABSTRACT

A safety syringe, including a syringe barrel, a needle holder, an injection needle, a ring plug, a push rod, a retractable rod, and a compression spring, where at least two guide grooves extending in an axial direction and passing through a retraction cavity are evenly distributed in an outer wall of the push rod along a circumferential direction; a stopper projecting portion protruding from a groove wall of each of the guide grooves is provided at a position of the guide groove close to a distal end. In the structure, based on the structures of the guide blocks and the stopper projecting portion, a larger breakthrough force is required during release of the potential energy of the compression spring, and there is almost no possibility of false triggering, ensuring better reliability.

### TECHNICAL FIELD

The present disclosure relates to the technical field of medical devices, and in particular to a safety syringe.

### BACKGROUND

A syringe is a widely used medical device. After injection, if an injection needle is exposed, it is easy to cause accidental injury to non-patients, and there is a very high risk of infection. For this reason, there is an increasing demand for safety syringes that can automatically hide injection needles after the injection is completed. An automatic retractable safety syringe can automatically retract an injection needle into a syringe barrel after the injection is completed, so as to prevent the injection needle from being exposed.

For example, the Chinese invention patent with publication number CN101284154B discloses a self-destructive safety syringe, which is an automatic retractable safety injector. According to the self-destructive safety syringe disclosed in this patent, after injection, a front end of a retractable rod is engaged with a tail end of a needle holder. After an elastic rubber ring breaks through the barrier of a limit ring, the potential energy of a retraction spring is released. Based on the potential energy release of the retraction spring, an assembly composed of the retractable rod, the needle holder, and a puncture needle tube is retracted into a barrel body of a push barrel, thereby providing protection for an injection needle.

### SUMMARY

### Technical Problems

However, the structure disclosed in the above-mentioned patent still has corresponding defects, specifically including the following technical defects:
(1) A retraction spring is sleeved over the retractable rod and located inside the barrel body of the push barrel, and therefore is located in the blind area of the field of vision during the installation, which makes installation more complicated and inefficient.
(2) In an initial state, the retractable rod remains in a corresponding state based on the obstruction of the elastic rubber ring by the limit ring. During this process, the potential energy of the retraction spring drives the elastic rubber ring to have a tendency to break through the limit ring. The elastic ring is usually a rubber ring, which is poor in reliability and has the risk of premature release.

### Technical Solutions

The technical problems to be solved by the present disclosure is to overcome the technical defects in the prior art that the installation position of the retraction spring is unreasonable and the reliability of the elastic rubber ring as a limiting structure is relatively poor.

In order to solve the above technical problems, a technical solution provided by the present disclosure is as follows: a safety syringe includes at least:
a syringe barrel, the syringe barrel being provided with an inner cavity extending from a proximal end to a distal end, a needle holder mounting portion being provided at the distal end of the inner cavity;
a needle holder, an injection needle being arranged at a distal end of the needle holder, a first engaging portion being provided at a proximal end of the needle holder, and the needle holder entering from the proximal end of the inner cavity and being mounted on the needle holder mounting portion;
a ring plug, an annular boss being provided at the proximal end of the needle holder, the ring plug being mounted between the annular boss and an inner wall of the inner cavity in an interference manner, an annular cavity for accommodating the ring plug being formed between the annular boss and the distal end of the inner cavity, and the ring plug being in clearance fit with the needle holder and the inner wall of the inner cavity in the annular cavity;
a push rod, the push rod being located inside the inner cavity, a retraction cavity being provided inside the push rod, a piston mounting portion available for mounting a piston being provided at a distal end of the push rod, and a step surface protruding from an outer wall of the push rod being provided on a proximal end side of the piston mounting portion;
a retractable rod, the retractable rod being mounted inside the retraction cavity, a second engaging portion for adapting to the first engaging portion being provided at an end portion of a distal end of the retractable rod; and
a compression spring,
where at least two guide grooves extending in an axial direction and passing through the retraction cavity are evenly distributed in the outer wall of the push rod along a circumferential direction; a stopper projecting portion protruding from a groove wall of each of the guide grooves is provided at a position of the guide groove close to a distal end; guide blocks in respective fit with the guide grooves are arranged at a proximal end of the retractable rod; the guide blocks extend from the retraction cavity along the guide grooves and protrude from the outer wall of the push rod; the compression spring is sleeved over the push rod and is located between the step surface and the guide blocks; and in an initial state, the guide blocks are located at distal end sides of the stopper projecting portions.

As a preferred embodiment, the inner wall of the inner cavity is provided with a first protrusion in the circumferential direction at a position corresponding to a proximal end side of the annular boss for preventing the ring plug from moving towards a proximal end.

As a preferred embodiment, both ends of an inner wall of the ring plug are provided with second protrusions protruding from the inner wall and covering both sides of the annular boss in the circumferential direction.

As a preferred embodiment, the first engaging portion includes an engaging groove extending from an end face of the proximal end of the needle holder to the distal end, and a first engaging convex portion is provided on an inner wall of the engaging groove.

As a preferred embodiment, a first guide surface is provided at an entrance of the engaging groove.

As a preferred embodiment, the second engaging portion includes at least two engaging arms arranged at intervals, a deformation gap is reserved between every two adjacent engaging arms, and second engaging convex portions interacting with the first engaging convex portion are provided at outer sides of the engaging arms.

As a preferred embodiment, a second guide surface is provided on an outer side of a distal end of each of the engaging arms.

As a preferred embodiment, a sealing ring is arranged between the distal end of the retractable rod and the retraction cavity.

As a preferred embodiment, an inwardly inclined sloping surface is provided on a distal end side of each of the guide blocks.

As a preferred embodiment, multiple cantilevers are arranged at the proximal end of the retractable rod, the guide blocks are arranged at free ends of the cantilevers, and guide sloping surfaces are provided on proximal end sides of the guide blocks.

As a preferred embodiment, the needle holder includes a holder body I connected to the syringe barrel and a holder body II detachably connected to a base, and the injection needle is fixedly connected to the holder body II.

As a preferred embodiment, a circumferential limiting structure is provided between the holder body I and the needle holder mounting portion.

### Beneficial Effects

Compared with the prior art, the safety syringe provided by the present disclosure has at least the following technical advantages.
(1) The push rod is provided with the guide grooves, the stopper projecting portions are provided on the groove walls of the guide grooves, the guide blocks protruding from the guide grooves are arranged on the retractable rod. In the initial state, the guide blocks are limited to the distal end side by the stopper projecting portions, and the compression spring is arranged between the piston mounting portion and the guide blocks. The premise of releasing the potential energy of the compression spring is that the guide blocks break through the restriction of the stopper projecting portions. During the breakthrough process, the stopper projecting portions, the guide grooves and the push rod wall need to be deformed integrally, so that a larger breakthrough force is required, there is almost no possibility of false triggering, and its reliability is far superior to the prior art.
(2) The compression spring is sleeved over the push rod, and is located between the piston mounting portion and the guide blocks. The installation process is visible, making installation more convenient and efficient, and location setting more reasonable.
(3) Due to arrangement of the guide grooves and the guide blocks, a guiding role is played in the retraction process of the retractable rod. The resistance encountered by the retractable rod in the retraction process is smaller, and the potential energy utilization efficiency of the compression spring is higher, so that the retraction speed is faster.

Another technical solution provided by the present disclosure is as follows: a safety syringe includes at least:
a syringe barrel, the syringe barrel being provided with an inner cavity extending from a proximal end to a distal end, a needle holder mounting portion being provided at the distal end of the inner cavity;
a needle holder, an injection needle being arranged at a distal end of the needle holder, a first engaging portion being provided at a proximal end of the needle holder, and the needle holder entering from the proximal end of the inner cavity and being mounted on the needle holder mounting portion;
a ring plug, an annular boss being provided at the proximal end of the needle holder, the ring plug being mounted between the annular boss and an inner wall of the inner cavity in an interference manner, an annular cavity for accommodating the ring plug being formed between the annular boss and the distal end of the inner cavity, and the ring plug being in clearance fit with the needle holder and the inner wall of the inner cavity in the annular cavity;
a push rod, the push rod being located inside the inner cavity, a retraction cavity being provided inside the push rod, a piston being mounted at a distal end of the push rod, a retractable rod coupling chamber being arranged at a distal end of the retraction cavity, a maximum aperture of the retractable rod coupling chamber being smaller than an aperture of the retraction cavity, and a first stopper portion being provided inside the retractable rod coupling chamber;
a retractable rod, including a retractable rod body that is in interference fit with the retractable rod coupling chamber, where a second stopper portion for interacting with the first stopper portion is provided at a proximal end side of the retractable rod body, a second engaging portion for adapting to the first engaging portion is provided at an end portion of a distal end of the retractable rod body, and a first spring mounting portion is provided at a proximal end of the retractable rod body; and
a tension spring, with one end being mounted on the first spring mounting portion, and the other end being fixedly connected to an end portion of a proximal end of the retraction cavity.

As a preferred embodiment, a transition conical surface is arranged between the retraction cavity and the retractable rod coupling chamber, and a sealing element for abutting against and sealing with the transition conical surface is arranged on the retractable rod.

As a preferred embodiment, the retractable rod body is conical, and an outer diameter of the retractable rod body gradually increases from the distal end to the proximal end. The retractable rod coupling chamber is conical, and an inner diameter of the retractable rod coupling chamber gradually increases from a distal end to the first stopper portion.

As a preferred embodiment, the inner wall of the inner cavity is provided with a first protrusion in a circumferential direction at a position corresponding to a proximal end side of the annular boss for preventing the ring plug from moving towards a proximal end.

As a preferred embodiment, the first engaging portion includes an engaging groove extending from an end face of the proximal end of the needle holder to the distal end, and a first engaging convex portion is provided on an inner wall of the engaging groove.

As a preferred embodiment, a first guide surface is provided at an entrance of the engaging groove.

As a preferred embodiment, the second engaging portion includes a clamping groove for accommodating the first engaging convex portion, and a clamping groove wall for limiting the first engaging convex portion within the clamping groove is provided at a distal end side of the clamping groove.

As a preferred embodiment, a conical guide portion is provided on a side of the clamping groove away from the retractable rod body.

As a preferred embodiment, a proximal end of the push rod is provided with a gland, the gland is provided with a second spring mounting portion, and a proximal end of the tension spring is connected to the second spring mounting portion.

As a preferred embodiment, the second stopper portion is provided with a guide surface.

According to the safety syringe in this embodiment, the first stopper portion is provided inside the retractable rod coupling chamber of the push rod, and the retractable rod is provided with the second stopper portion interacting with the first stopper portion. Based on the interference fit between the retractable rod body and the retractable rod coupling chamber as well as the arrangement of the first stopper portion and the second stopper portion, the initial limit of the retractable rod is achieved, and the sealing between the retractable rod body and the retractable rod coupling chamber is realized. In addition, since the retractable rod is usually an injection molded member, it may be deformed under a higher pushing force, which makes the retractable rod pass over the first stopper portion. In this initial limiting way, there is no need to set a guide groove on the push rod, so that the diameter of the push rod may be made smaller on the premise of ensuring the rigidity of the push rod; and correspondingly, the diameter of the syringe may also be made smaller, thereby meeting the injection demand of small doses of liquid medicine.

### BRIEF DESCRIPTION OF THE DRAWINGS

FIG. 1 is a schematic structural diagram of a safety syringe illustrated in Embodiment I;

FIG. 2 is a schematic structural diagram of the safety syringe illustrated in Embodiment I in an exploded state;

FIG. 3 is a schematic structural diagram of a syringe barrel in the safety syringe illustrated in Embodiment I;

FIG. 4 is a schematic structural diagram of a push rod, a retractable rod and a compression spring in the safety syringe illustrated in Embodiment I in an assembled state;

FIG. 5 is a schematic structural diagram of the structure shown in FIG. 4 in a cross-sectional state;

FIG. 6 is a schematic structural diagram of the push rod in the safety syringe illustrated in Embodiment I;

FIG. 7 is a schematic diagram of the three-dimensional structure of the retractable rod in the safety syringe illustrated in Embodiment I;

FIG. 8 is a schematic structural diagram of the retractable rod shown in FIG. 7 in a front view state;

FIG. 9 is a schematic structural diagram of a needle holder and an injection needle in a first structural form in the safety syringe illustrated in Embodiment I;

FIG. 10 is a schematic diagram of the partial cross-sectional structure of the needle holder in FIG. 9;

FIG. 11 is a schematic structural diagram of the needle holder and the injection needle in a second structural form in the safety syringe illustrated in Embodiment I;

FIG. 12 is a schematic diagram of the partial cross-sectional structure of the safety syringe illustrated in Embodiment I in an initial state;

FIG. 13 is a schematic structural diagram of the safety syringe illustrated in Embodiment I after absorbing liquid medicine;

FIG. 14 is a schematic structural diagram of the safety syringe illustrated in Embodiment I after liquid medicine injection is completed;

FIG. 15 is a schematic structural diagram of the safety syringe illustrated in Embodiment I after continuing to apply pressure to the push rod based on the state shown in FIG. 14 until the needle holder is engaged with the retractable rod;

FIG. 16 is a schematic structural diagram of the safety syringe illustrated in Embodiment I after continuing to apply pressure to the push rod based on the state shown in FIG. 15 until a ring plug enters an annular cavity and guide blocks break through the restriction of stopper projecting portions;

FIG. 17 is a schematic structural diagram of an assembly composed of the retractable rod, the needle holder, and the injection needle in the safety syringe illustrated in Embodiment I in a state where the assembly is retracted into an interior of the syringe barrel;

FIG. 18 is a schematic diagram of the partial structure of a safety syringe illustrated in Embodiment II in an initial state;

FIG. 19 is a partially enlarged schematic diagram of Part A shown in FIG. 18;

FIG. 20 is a schematic diagram of the partial structure of a syringe barrel in the safety syringe illustrated in Embodiment II;

FIG. 21 is a partially enlarged schematic diagram of Part B shown in FIG. 20;

FIG. 22 is a schematic structural diagram of a ring plug in the safety syringe illustrated in Embodiment II;

FIG. 23 is a schematic structural diagram of a safety syringe illustrated in Embodiment III in an exploded state;

FIG. 24 is a schematic structural diagram of a syringe barrel in the safety syringe illustrated in Embodiment III;

FIG. 25 is a schematic diagram of the cross-sectional structure of a push rod in the safety syringe illustrated in Embodiment III;

FIG. 26 is a schematic structural diagram of a retractable rod in the safety syringe illustrated in Embodiment III;

FIG. 27 is a schematic structural diagram of the retractable rod shown in FIG. 26 in a state of being assembled with a sealing element;

FIG. 28 is a schematic structural diagram of a needle holder and an injection needle in the safety syringe illustrated in Embodiment III in a combined state;

FIG. 29 is a schematic diagram showing the combination of the push rod and the retractable rod in the safety syringe illustrated in Embodiment III in an initial state;

FIG. 30 is a schematic structural diagram of the safety syringe illustrated in Embodiment III, in which the retractable rod and the needle holder are in a separated state;

FIG. 31 is a schematic structural diagram of the safety syringe illustrated in Embodiment III, in which the retractable rod and the needle holder are in a combined state; and

FIG. 32 is a schematic structural diagram of the safety syringe illustrated in Embodiment III, in which an assembly composed of the needle holder, the injection needle, and the retractable rod is located inside a retraction cavity.

### BEST EMBODIMENT OF THE INVENTION

Enter the description paragraphs of the best embodiment of the present disclosure here.

### DETAILED DESCRIPTION

In order to make the objectives, technical solutions, and advantages of the present disclosure clearer, the present disclosure will be further described in detail below in conjunction with the accompanying drawings and embodiments. It is to be understood that specific embodiments described herein are only adopted to explain the present disclosure and not intended to limit the present disclosure.

In the description of the present disclosure, it should be understood that the orientations or positional relationships indicated by the terms such as “upper”, “lower”, “front”, “back”, “inner”, and “outer” are based on the orientations or positional relationships shown in the drawings, which are only for convenience in describing the present disclosure and simplifying the description, and do not indicate or imply that the devices or elements referred to must have particular orientations, or must be constructed and operated in particular orientations. Therefore, it cannot be construed as a limitation on the present disclosure.

In the description of the present disclosure, it should be noted that unless otherwise explicitly specified or defined, the terms such as “mount”, “connect”, and “connection” should be understood in a broad sense. For example, the connection may be a fixed connection, an integral connection, or a detachable connection; or the connection may be an internal communication between two components; or the connection may be a direct connection, or an indirect connection through an intermediary. Those of ordinary skill in the art could understand the specific meanings of the above terms in the present disclosure according to specific situations.

In the description of the present disclosure, it should be noted that an end of a syringe close to a user is set as a proximal end, and an end close to a patient is set as a distal end.

### Embodiment I

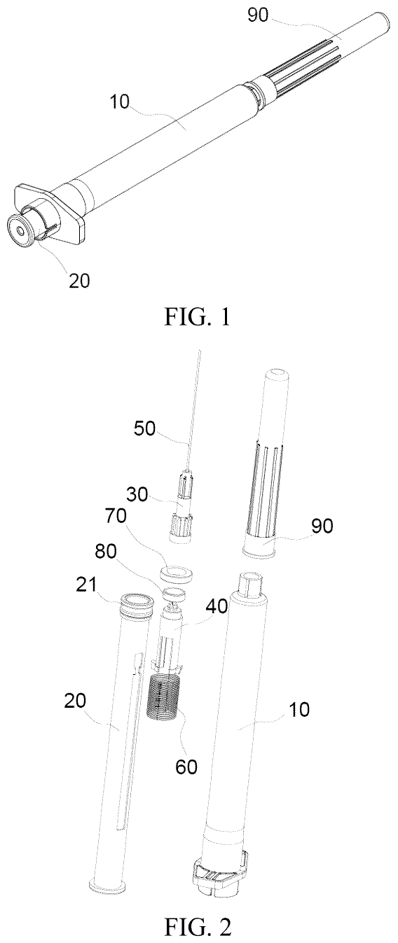

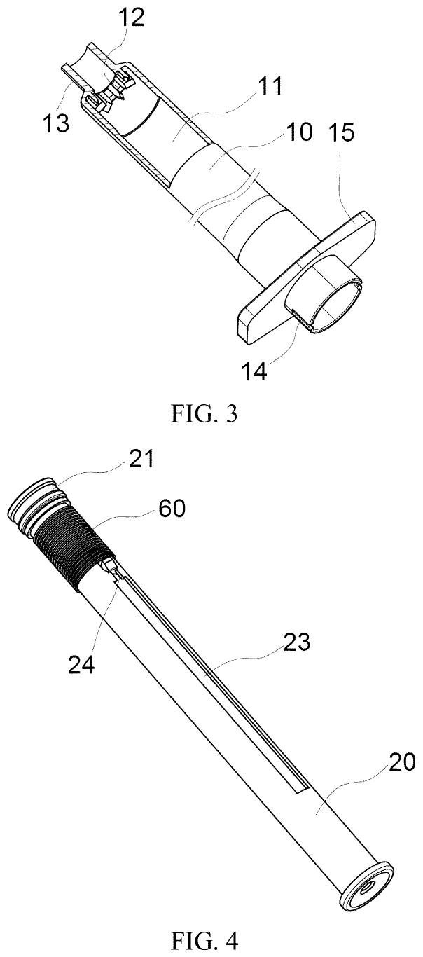

A safety syringe of this embodiment, as shown in FIG. 1 and FIG. 2, includes a syringe barrel 10, a push rod 20, a needle holder 30, a retractable rod 40, an injection needle 50, a compression spring 60, and a protective cap 90, where the injection needle 50 is fixedly mounted at a distal end of the needle holder 30, as shown in FIG. 3; a distal end of the syringe barrel 10 is provided with a protective cap coupling seat 13 for matching the protective cap; and in an initial state, the protective cap 90 is sleeved over the injection needle 50 and in fit connection with the protective cap coupling seat 13.

In this embodiment, as shown in FIG. 3, the syringe barrel 10 is provided with an inner cavity 11 extending from a proximal end to a distal end, and the push rod 20 is located inside the inner cavity 11.

In this embodiment, a needle holder mounting portion 12 is provided at the distal end of the inner cavity 11, as shown in FIG. 12, the needle holder 30 enters from the proximal end of the inner cavity and is limited to the needle holder mounting portion 12 by a ring plug 70. Specifically, as shown in FIG. 9, an annular boss 31 is provided at the proximal end of the needle holder, and as shown in FIG. 12, the ring plug 70 is mounted between the annular boss 31 and an inner wall of the inner cavity 11 in an interference manner. It should be noted that the ring plug has a certain degree of elasticity and can be axially displaced under the action of an external force. Specifically, in this embodiment, an annular cavity 26 for accommodating the ring plug 70 is formed between the annular boss 31 and the distal end of the inner cavity 11, and the ring plug 70 is in clearance fit with the needle holder 30 and the inner wall of the inner cavity 11 in the annular cavity 26.

In this embodiment, the syringe barrel 10 is provided with a handheld operating portion 15 at a position near the proximal end, which is a conventional structure of a syringe. As a special feature of this embodiment, as shown in FIG. 3, the syringe barrel 10 may be provided with multiple through grooves 14 that extend from an end face of the proximal end to the handheld operating portion 15 and penetrate through a wall of the syringe barrel.

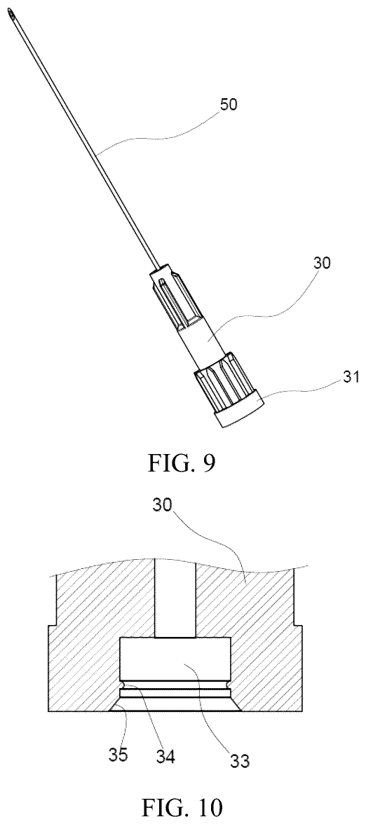

In this embodiment, the needle holder 30 of a first structural form has a structure as shown in FIG. 9 and FIG. 10, and a first engaging portion is provided at the proximal end of the needle holder. Preferably, in this embodiment, the first engaging portion includes an engaging groove 33 extending from an end face of the proximal end of the needle holder 30 to the distal end, and a first engaging convex portion 34 is provided on an inner wall of the engaging groove 33.

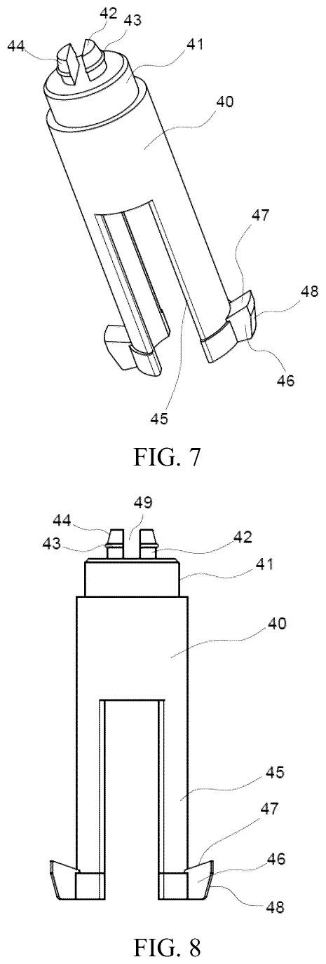

In this embodiment, as shown in FIG. 7 and FIG. 8, a second engaging portion for adapting to the first engaging portion is provided at an end portion of a distal end of the retractable rod 40. Preferably, in this embodiment, the second engaging portion includes two engaging arms 42 arranged at intervals, a deformation gap 49 is reserved between the two adjacent engaging arms 42, and second engaging convex portions 43 interacting with the first engaging convex portion 34 are provided at outer sides of the engaging arms 42.

When the distal end of the retractable rod 40 moves towards the proximal end of the needle holder 30 under the action of an external force, the multiple engaging arms 42 enter the engaging groove 33. Based on the setting of the deformation gap 49, the engaging arms 42 are deformed to a certain extent, so that the second engaging convex portions 43 pass through the position where the first engaging convex portion 34 is located. In this case, the deformation of the engaging arms 42 is restored, and the retractable rod 40 and the needle holder 30 are engaged into an assembly based on the blocking of the second engaging convex portions 43 by the first engaging convex portion 34.

It should be noted that the retractable rod is usually an injection molded member, and its engaging arms 42 have certain deformation characteristics.

Preferably, in this embodiment, a first guide surface 35 is provided at an entrance of the engaging groove 33, and correspondingly, a second guide surface 44 is provided on an outer side of a distal end of each of the engaging arms 42. Based on the interaction between the first guide surface 35 and the second guide surface 44, the engagement between the retractable rod and the needle holder is more labor-saving and smoother.

It should be noted that the specific structures of the first engaging portion and the second engaging portion provided in this embodiment are the preferred implementation manners of this embodiment, and the first engaging portion and the second engaging portion may also be engaged using any suitable engaging structure in the prior art.

The structure of the push rod 20 in this embodiment is shown in FIG. 4 to FIG. 6, a retraction cavity 22 is provided inside the push rod, a piston mounting portion 25 available for mounting a piston 21 is provided at a distal end of the push rod, and a step surface 26 protruding from an outer wall of the push rod is provided on a proximal end side of the piston mounting portion 25.

As a special feature of this embodiment, two guide grooves 23 extending in an axial direction and passing through the retraction cavity 22 are evenly distributed in the outer wall of the push rod 20 along a circumferential direction, and a stopper projecting portion 24 protruding from a groove wall of each of the guide grooves is provided at a position of the guide groove 23 close to a distal end. Preferably, in this embodiment, a pair of stopper projecting portions 24 are arranged opposite to each other.

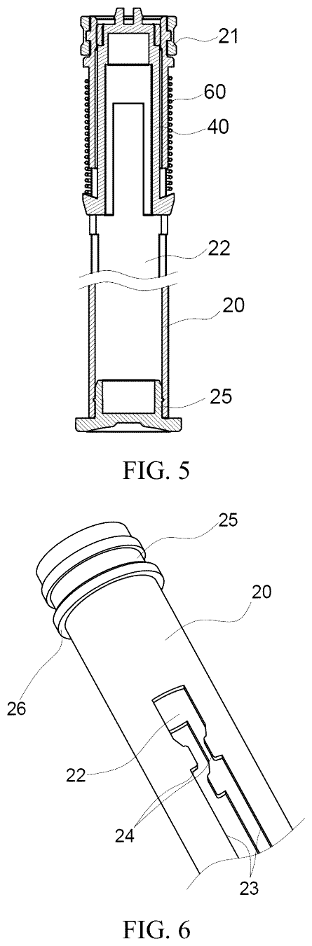

The structure of the retractable rod 40 in this embodiment is shown in FIG. 7 and FIG. 8. Two cantilevers 45 are arranged at the proximal end of the retractable rod, and end portions of free ends of the cantilevers 45 are provided with guide blocks 46 in respective fit with the guide grooves 23. As shown in FIG. 4 and FIG. 5, the guide blocks 46 extend from the retraction cavity 21 along the guide grooves 23 and protrude from the outer wall of the push rod 20, and in the initial state, the guide blocks 46 are located at distal end sides of the stopper projecting portions 24, thereby limiting the retractable rod to the distal end position of the retraction cavity.

As a special feature of this embodiment, as shown in FIG. 4 and FIG. 5, the compression spring 60 is sleeved over the push rod 20 and is located between a step surface 26 and the guide blocks 46.

Preferably, as shown in FIG. 7 and FIG. 8, an inwardly inclined sloping surface 47 is provided on a distal end side of each of the guide blocks 46. The purpose of this arrangement is to prevent the compression spring 60 from falling off from the guide blocks 46.

Preferably, as shown in FIG. 7 and FIG. 8, guide sloping surfaces 48 are provided on proximal end sides of the guide blocks 46, which functions to guide the deformation of the cantilevers 45 when the guide blocks 46 are installed from the distal end of the push rod 20 into the retraction cavity.

Preferably, as shown in FIG. 4 to FIG. 8, a sealing groove 41 is formed at the distal end of the retractable rod 40 for installation of a sealing ring 80. The sealing ring 80 is arranged between the distal end of the retractable rod 40 and the retraction cavity to provide a sealing effect, which prevents liquid medicine from entering the retraction cavity. It should be noted that in this embodiment, preferably, the distal end of the sealing groove 41 is set in an open manner, and the sealing ring 80 is preferably a rectangular sealing ring.

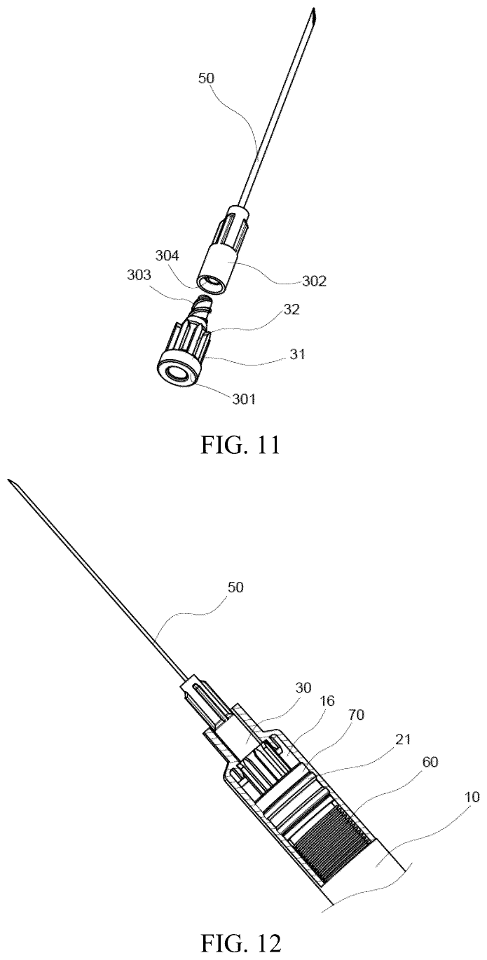

In this embodiment, a needle holder of a second structural form is provided, as shown in FIG. 11, the needle holder includes a holder body I 301 connected to the syringe barrel and a holder body II 302 detachably connected to a base, and the injection needle is fixedly connected to the holder body II 302. The purpose of this arrangement is to achieve the effect of changing the size of the injection needle. Generally, a syringe body belongs to a finished product, and its holder body I has been fixedly mounted in the syringe barrel. During injection, injection needles of different sizes are arranged on corresponding holder bodies II, and a doctor can choose the appropriate size of injection needle according to the need and connect same to the holder body I.

Preferably, in this embodiment, the holder body I and the holder body II are connected by Luer tapers commonly used in the field of medical devices, the distal end of the holder body I is a male taper, and a proximal end of the holder body II is a female taper. It should be noted that this is only a preferred implementation manner of this embodiment, and other forms of separable connection methods may also be adopted.

In this embodiment, the Luer tapers are operated by rotation. Therefore, preferably, in this embodiment, a circumferential limiting structure is provided between the holder body I and the needle holder mounting portion 12. Specifically, a slotted structure 32 is provided on the holder body I at a position adjacent to the annular boss 31, and correspondingly, the needle holder mounting portion 12 is provided with a structure adapted to the slotted structure 32. In this way, during the rotational engagement of the holder body II and the holder body I, the holder body I is prevented from rotating relative to the syringe barrel.

It should be noted that when the needle holder 30 adopts the first implementation manner shown in FIG. 9 and FIG. 10, the circumferential limiting structure may be provided, or the circumferential limiting structure may not be provided.

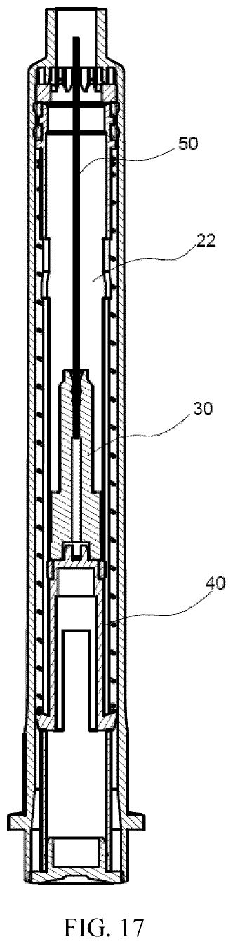

Preferably, in this embodiment, as shown in FIG. 8, a pressing end cap 25 is arranged at a proximal end of the push rod 20, and as shown in FIG. 17, in a retraction completion state after injection, based on the arrangement of the through grooves 14, the pressing end cap 25 can enter the inner cavity of the syringe barrel, so that the pressing end cap and the syringe barrel can be combined together to prevent repeated insertion and removal of the push rod.

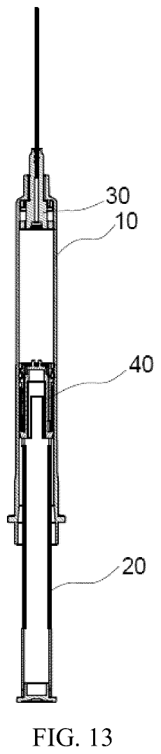

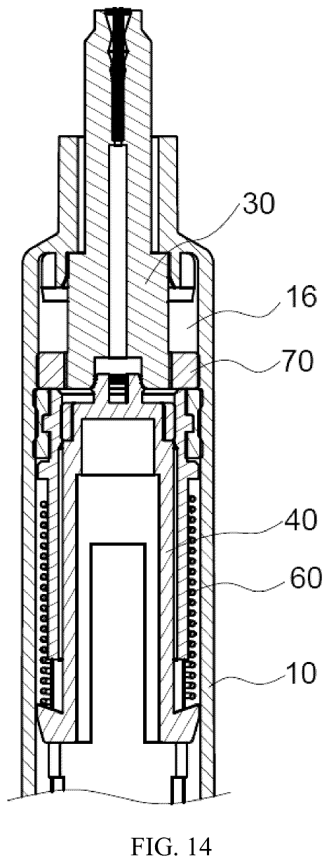

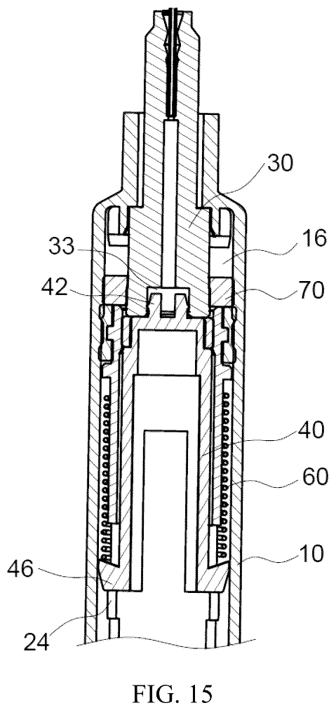

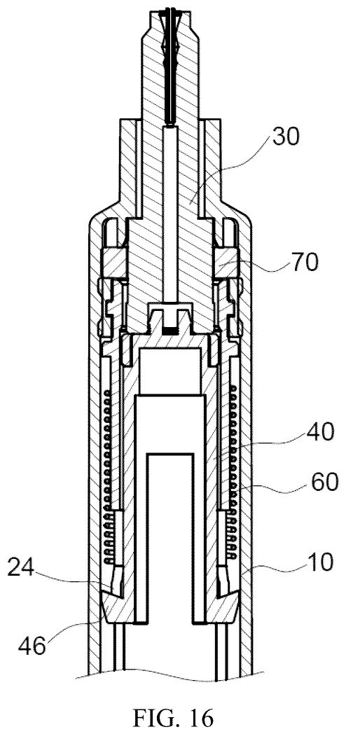

The working principle of the safety syringe in this embodiment is as follows:
(1) In the initial state shown in FIG. 1, the protective cap 90 is pulled out, the internal structure is as shown in FIG. 12, and the distal end of the piston 21 on the push rod 20 is in contact with the proximal end of the needle holder.
(2) The push rod is pulled to absorb liquid medicine, as shown in FIG. 13.
(3) The push rod 20 is pushed, and liquid medicine injection is completed, as shown in FIG. 14. In this case, a distal end of the push rod contacts a proximal end of the ring plug 70.
(4) The push rod is continued to be pushed, as shown in FIG. 15, so that the second engaging portion of the retractable rod 40 engages with the first engaging portion of the needle holder. During this process, an end portion of a distal end of the push rod acts on an end face of the proximal end of the ring plug, driving the ring plug to move into the annular cavity 26.
(5) After the second engaging portion of the retractable rod 40 is engaged with the first engaging portion of the needle holder, as shown in FIG. 16, the push rod 20 is continued to be pushed. In this case, the position of the retractable rod 40 is relatively fixed. In the process of the push rod continuing to move, the ring plug is pushed into the annular cavity 26. In this case, the ring plug releases the interference fit between the needle holder and the inner cavity. Moreover, the guide blocks 46 drive the deformation of the stopper projecting portions 24 based on the relative motion between the push rod and the retractable rod, thereby releasing the restriction on the guide blocks 46 and allowing the guide blocks 46 to move to proximal end sides of the stopper projecting portions 24.
(6) After the stopper projecting portions 24 release the restriction on the guide blocks 46, the potential energy of the compression spring 60 is released, and the assembly composed of the retractable rod, the needle holder and the injection needle is driven to enter the interior of the syringe barrel until the state shown in FIG. 17, so as to realize the automatic protection of the injection needle.

### Embodiment II

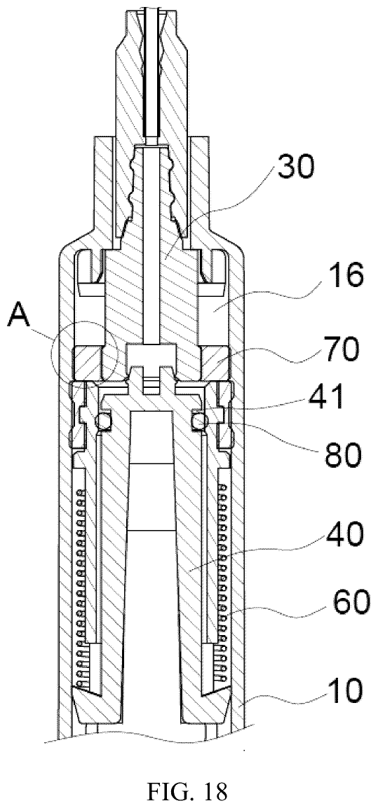

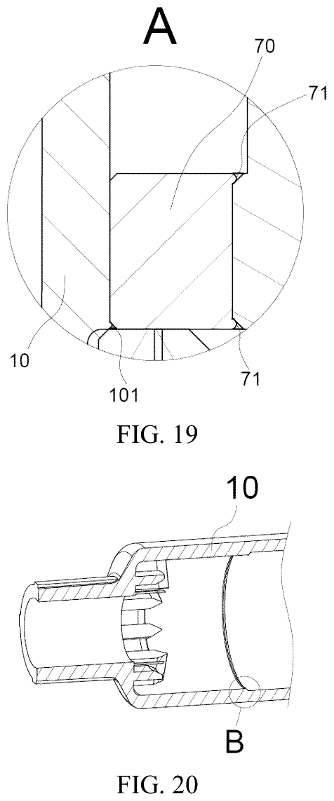

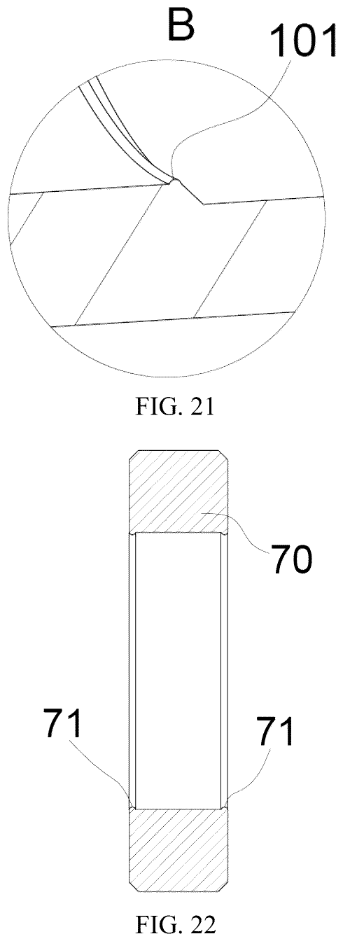

As shown in FIG. 18 to FIG. 22, a safety syringe of this embodiment is different from Embodiment I in that, based on the structure of Embodiment I, the inner wall of the inner cavity 11 in the syringe barrel 10 is provided with a first protrusion 101 in the circumferential direction at a position corresponding to a proximal end side of the annular boss 31 in the needle holder 30 for preventing the ring plug 70 from moving towards the proximal end.

It should be noted that, in this embodiment, the first protrusion 101 may be continuous along the circumferential direction, or may include multiple protrusions distributed at intervals along the circumferential direction. Preferably, the first protrusion 101 is continuous along the circumferential direction.

In this embodiment, the purpose of setting the first protrusion 101 is that when the injection needle 50 of the safety syringe enters a liquid medicine bottle, the needle holder will have a tendency to move towards the proximal end, and the first protrusion 101 forms a limit to prevent the assembly composed of the needle holder and the ring plug from moving towards the proximal end.

Further, preferably, in this embodiment, based on the structure of Embodiment I, both ends of an inner wall of the ring plug 70 are provided with second protrusions 71 protruding from the inner wall and covering both sides of the annular boss 31 in the circumferential direction.

It should be noted that, in this embodiment, the second protrusions 71 may be continuous along the circumferential direction, or may include multiple protrusions distributed at intervals along the circumferential direction. Preferably, the second protrusions 71 are continuous along the circumferential direction.

In this embodiment, the purpose of setting the second protrusions 71 is that when the injection needle 50 of the safety syringe enters a liquid medicine bottle, the needle holder will have a tendency to move towards the proximal end, and the second protrusions 71 form a limit to prevent the needle holder from separating from the ring plug.

Preferably, in this embodiment, a sealing groove 41 is formed at the distal end of the retractable rod 40 for installation of the sealing ring 80. The sealing ring 80 is arranged between the distal end of the retractable rod 40 and the retraction cavity to provide a sealing effect, which prevents liquid medicine from entering the retraction cavity. It should be noted that in this embodiment, preferably, the difference between this embodiment and Embodiment I is that both a distal end and a proximal end of the sealing groove 41 are closed, and the sealing ring 80 is preferably an O-shaped sealing ring.

The working principle of the safety syringe of this embodiment is the same as that of Embodiment I, and will not be repeated herein.

### Embodiment III

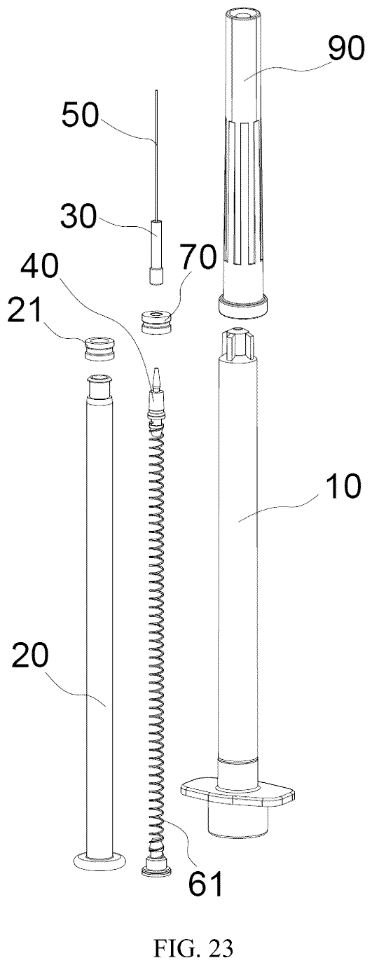

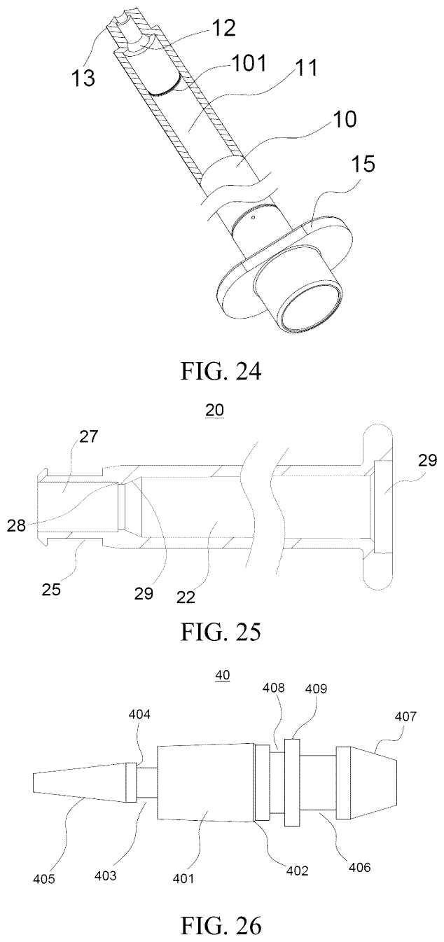

A safety syringe of this embodiment, as shown in FIG. 23, includes a syringe barrel 10, a push rod 20, a needle holder 30, a retractable rod 40, an injection needle 50, a tension spring 61, and a protective cap 90, where the injection needle 50 is fixedly mounted at a distal end of the needle holder 30, as shown in FIG. 24; a distal end of the syringe barrel 10 is provided with a protective cap coupling seat 13 for matching the protective cap 90; and in an initial state, the protective cap 90 is sleeved over the injection needle 50 and in fit connection with the protective cap coupling seat 13.

In this embodiment, as shown in FIG. 24, the syringe barrel 10 is provided with an inner cavity 11 extending from a proximal end to a distal end, and the push rod 20 is located inside the inner cavity 11. A needle holder mounting portion 12 is provided at the distal end of the inner cavity 11. After entering the inner cavity 11 from the proximal end of the syringe barrel 10, the needle holder 30 is limited to the needle holder mounting portion 12 by a ring plug 70.

Preferably, in this embodiment, an inner wall of the inner cavity 11 in the syringe barrel 10 is provided with a first protrusion 101 in the circumferential direction at a position corresponding to the proximal end side of an annular boss 31 in the needle holder 30 for preventing the ring plug 70 from moving towards the proximal end.

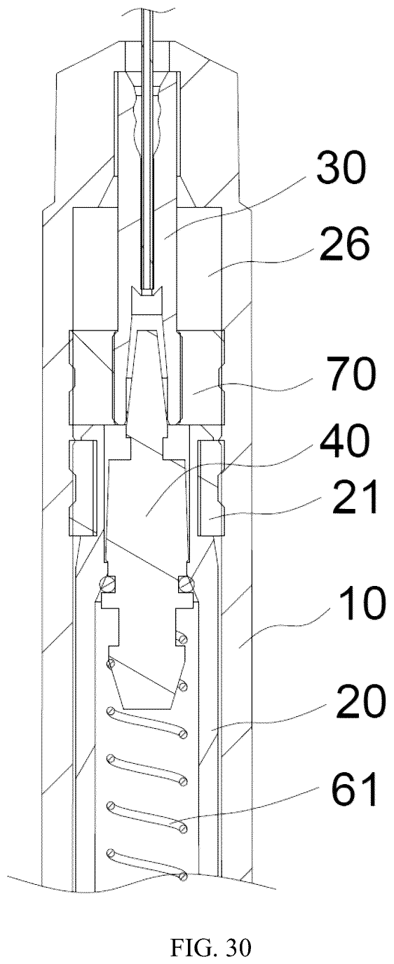

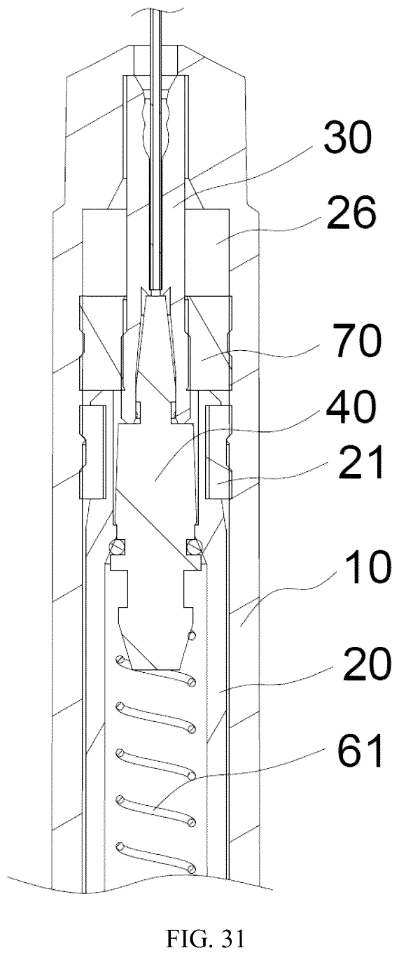

Specifically in this embodiment, as shown in FIG. 28, an annular boss 31 is provided at the proximal end of the needle holder, and as shown in FIG. 30 and FIG. 31, the ring plug 70 is mounted between the annular boss 31 and the inner wall of the inner cavity 11 in an interference manner. It should be noted that the ring plug has a certain degree of elasticity and can be axially displaced under the action of an external force. Specifically in this embodiment, an annular cavity 26 for accommodating the ring plug 70 is formed between the annular boss 31 and the distal end of the inner cavity 11, and the ring plug 70 is in clearance fit with the needle holder 30 and the inner wall of the inner cavity 11 in the annular cavity 26.

In this embodiment, as shown in FIG. 28, the injection needle 50 is fixedly arranged at the distal end of the needle holder 30, and a first engaging portion is provided at the proximal end of the needle holder 30. Preferably, in this embodiment, the first engaging portion includes an engaging groove 33 extending from an end face of the proximal end of the needle holder 30 to the distal end, a first engaging convex portion 34 is provided on an inner wall of the engaging groove 33, and a first guide surface 35 is provided at an entrance of the engaging groove 33.

In this embodiment, as shown in FIG. 25, a retraction cavity 22 is provided inside the push rod 20, and a piston mounting portion 25 available for mounting a piston 21 is provided at the distal end of the push rod. As a special feature of this embodiment, a retractable rod coupling chamber 27 is arranged at a distal end of the retraction cavity 22, a maximum aperture of the retractable rod coupling chamber is smaller than an aperture of the retraction cavity, and a first stopper portion 28 is provided inside the retractable rod coupling chamber 27. Preferably, in the present embodiment, the first stopper portion 28 is a step.

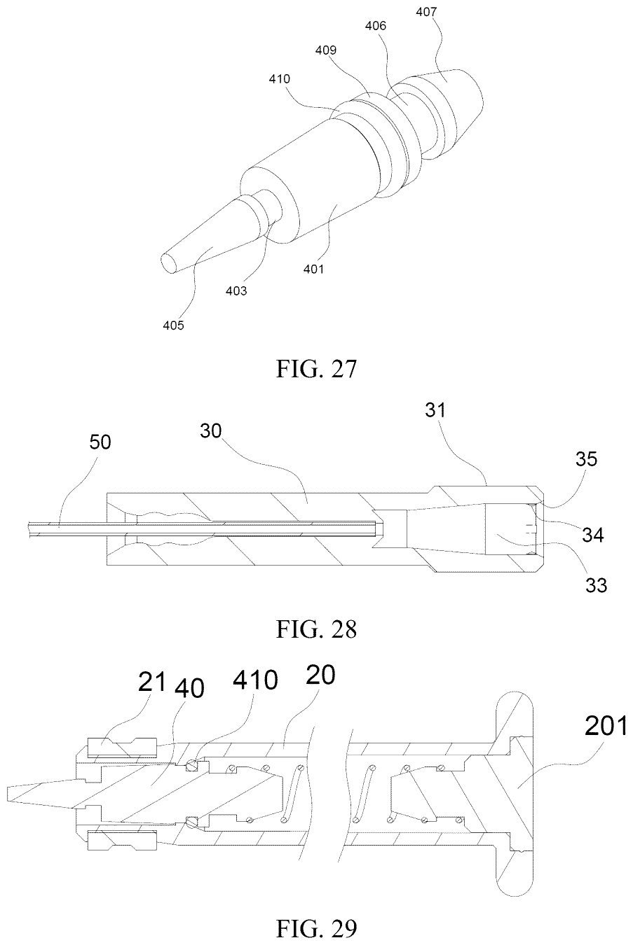

In this embodiment, as shown in FIG. 26 and FIG. 27, the retractable rod 40 includes a retractable rod body 401 that is in interference fit with the retractable rod coupling chamber 27, and a second stopper portion 402 for interacting with the first stopper portion 28 is provided at a proximal end side of the retractable rod body 401.

In this embodiment, based on the interference fit between the retractable rod body and the retractable rod coupling chamber as well as the arrangement of the first stopper portion and the second stopper portion, the initial limit of the retractable rod is achieved, and the sealing between the retractable rod body and the retractable rod coupling chamber is realized.

Generally, the retractable rod is an injection molded member, which can be deformed under the action of a large external force, so that the retractable rod can pass over the first stopper portion. Preferably, in this embodiment, the second stopper portion is provided with a guide surface to facilitate deformation of the retractable rod and passage over the first stopper portion.

In this embodiment, in order to further improve the sealing reliability between the retractable rod and the retractable rod coupling chamber, the retractable rod is provided with a sealing element 410 for abutting against and sealing with a transition conical surface 29 arranged between the retraction cavity and the retractable rod coupling chamber. In this embodiment, a sealing groove 408 is formed in the retractable rod, and a stopper boss 409 is provided on the side of the sealing groove away from the second stopper portion 402. The sealing element 410 is mounted inside the sealing groove 408.

It should be noted that the mounting manner of the sealing element is a preferred implementation manner of this embodiment, and as an equivalent implementation manner, the sealing element may also be integrally formed by injection molding with the retractable rod.

Preferably, in this embodiment, the retractable rod body 401 is tapered, and an outer diameter of the retractable rod body 401 gradually increases from the distal end to the proximal end.

In this embodiment, a second engaging portion for adapting to the first engaging portion is provided at an end portion of a distal end of the retractable rod body 401. Specifically in this embodiment, the second engaging portion includes a clamping groove 403 for accommodating the first engaging convex portion 34, and a clamping groove wall 404 for limiting the first engaging convex portion 34 within the clamping groove is provided at a distal end side of the clamping groove 403. Preferably, a conical guide portion 405 is provided on a side of the clamping groove 403 away from the retractable rod body 401.

Preferably, in this embodiment, a first spring mounting portion is provided at a proximal end of the retractable rod body 401. Specifically in this embodiment, the first spring mounting portion includes a mounting groove 406, and a guide taper portion 407 is provided on the proximal end side of the mounting groove 406, which facilitates the entering of an end portion of the tension spring 61 into the mounting groove 406.

In this embodiment, one end of the tension spring 61 is mounted on the first spring mounting portion, and the other end thereof is fixedly connected to an end portion of a proximal end of the retraction cavity. Specifically in this embodiment, as shown in FIG. 25, a gland mounting groove 29 is formed at the proximal end side of the retraction cavity 22. As shown in FIG. 29, a gland 201 is mounted inside the gland mounting groove 29, the gland 201 is provided with a second spring mounting portion, and a proximal end of the tension spring 61 is connected to the second spring mounting portion.

The working principle of the safety syringe in this embodiment is as follows:

The initial state is shown in FIG. 30, where the needle holder 30 is fixed in the syringe barrel by the ring plug 70, the retractable rod 40 is fixed in the retractable rod coupling chamber 27 by interference fit, and the tension spring 61 is in a tension energy storage state. In this state, liquid medicine can be adsorbed and injected by pulling and pushing the push rod 20.

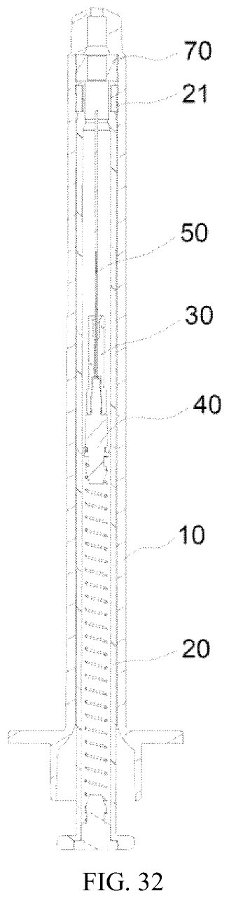

After the liquid medicine injection is completed, as shown in FIG. 31, a pushing force is applied to the push rod 20, so that the first engaging portion provided at the proximal end of the needle holder 30 engages with the second engaging portion provided at the distal end of the retractable rod 40, and the needle holder and the retractable rod are combined into an assembly. Then, a pushing force is continuously applied to the push rod 20. In this case, because the assembly formed by the needle holder and the retractable rod cannot move further to the distal end, the push rod 20 moves axially relative to the retractable rod 40, the retractable rod is deformed under the action of the pushing force, and the second stopper portion of the retractable rod passes over the first stopper portion in the retractable rod coupling chamber, thereby releasing the limit on the retractable rod; moreover, the distal end of the push rod pushes the ring plug 70 to move towards the interior of the annular cavity 26 until the ring plug 70 enters the annular cavity 26, and therefore the fixation of the needle holder 30 is released. In this state, under the potential energy of the tension spring, as shown in FIG. 32, the assembly composed of the needle holder, the injection needle and the retractable rod is pulled into the retraction cavity 22.

It should be noted that, according to the safety syringe shown in Embodiment I and Embodiment II, since the guide grooves are provided in the push rod, in order to ensure the rigidity of the push rod, the diameter of the push rod is larger, and therefore the diameter of the syringe is relatively large, making the syringe suitable for injection of a large dose of liquid medicine; and it is difficult to achieve the demand of injection of a small dose of liquid medicine (for example, injection of liquid medicine at a dose of 1 ml or less).

However, according to the safety syringe shown in Embodiment III, the first stopper portion is provided inside the retractable rod coupling chamber of the push rod, and the second stopper portion interacting with the first stopper portion is provided on the retractable rod. Based on the interference fit between the retractable rod body and the retractable rod coupling chamber as well as the arrangement of the first stopper portion and the second stopper portion, the initial limit of the retractable rod is achieved, and the sealing between the retractable rod body and the retractable rod coupling chamber is realized. In addition, since the retractable rod is usually an injection molded member, it may be deformed under a higher pushing force, which makes the retractable rod pass over the first stopper portion. In this initial limiting way, compared with Embodiment I and Embodiment II, there is no need to set a guide groove on the push rod, so that the diameter of the push rod may be made smaller on the premise of ensuring the rigidity of the push rod; and correspondingly, the diameter of the syringe may also be made smaller, thereby meeting the injection demand of small doses of liquid medicine.

In summary, the foregoing descriptions are merely exemplary embodiments of the present disclosure, and are not intended to limit the present disclosure. Any modifications, equivalent substitutions, and improvements made within the spirit and principles of the present disclosure shall be included within the scope of protection of the present disclosure.

### INDUSTRIAL APPLICABILITY

Enter the description paragraphs of industrial applicability here.

### Free Content of Sequence Listing

Enter the free description paragraphs of sequence listing here.

1-22. (canceled)

23. A safety syringe, comprising:
a syringe barrel, the syringe barrel being provided with an inner cavity extending from a proximal end to a distal end, a needle holder mounting portion being provided at the distal end of the inner cavity;
a needle holder, an injection needle being arranged at a distal end of the needle holder, a first engaging portion being provided at a proximal end of the needle holder, and the needle holder entering from the proximal end of the inner cavity and being mounted on the needle holder mounting portion;
a ring plug, an annular boss being provided at the proximal end of the needle holder, the ring plug being mounted between the annular boss and an inner wall of the inner cavity in an interference manner, an annular cavity for accommodating the ring plug being formed between the annular boss and the distal end of the inner cavity, and the ring plug being in clearance fit with the needle holder and the inner wall of the inner cavity in the annular cavity;
a push rod, the push rod being located inside the inner cavity, a retraction cavity being provided inside the push rod, a piston mounting portion available for mounting a piston being provided at a distal end of the push rod, and a step surface protruding from an outer wall of the push rod being provided on a proximal end side of the piston mounting portion;
a retractable rod, the retractable rod being mounted inside the retraction cavity, a second engaging portion for adapting to the first engaging portion being provided at an end portion of a distal end of the retractable rod; and
a compression spring,
wherein at least two guide grooves extending in an axial direction and passing through the retraction cavity are evenly distributed in the outer wall of the push rod along a circumferential direction; a stopper projecting portion protruding from a groove wall of each of the guide grooves is provided at a position of the guide groove close to a distal end; guide blocks in respective fit with the guide grooves are arranged at a proximal end of the retractable rod; the guide blocks extend from the retraction cavity along the guide grooves and protrude from the outer wall of the push rod; the compression spring is sleeved over the push rod and is located between the step surface and the guide blocks; and in an initial state, the guide blocks are located at distal end sides of the stopper projecting portions.

24. The safety syringe according to claim 23, wherein the inner wall of the inner cavity is provided with a first protrusion in the circumferential direction at a position corresponding to a proximal end side of the annular boss for preventing the ring plug from moving towards a proximal end.

25. The safety syringe according to claim 23, wherein both ends of an inner wall of the ring plug are provided with second protrusions protruding from the inner wall and covering both sides of the annular boss in the circumferential direction.

26. The safety syringe according to claim 24, wherein both ends of an inner wall of the ring plug are provided with second protrusions protruding from the inner wall and covering both sides of the annular boss in the circumferential direction.

27. The safety syringe according to claim 23, wherein the first engaging portion comprises an engaging groove extending from an end face of the proximal end of the needle holder to the distal end, and a first engaging convex portion is provided on an inner wall of the engaging groove.

28. The safety syringe according to claim 27, wherein a first guide surface is provided at an entrance of the engaging groove.

29. The safety syringe according to claim 27, wherein the second engaging portion comprises at least two engaging arms arranged at intervals, a deformation gap is reserved between every two adjacent engaging arms, and second engaging convex portions interacting with the first engaging convex portion are provided at outer sides of the engaging arms.

30. The safety syringe according to claim 29, wherein a second guide surface is provided on an outer side of a distal end of each of the engaging arms.

31. The safety syringe according to claim 28, wherein the second engaging portion comprises at least two engaging arms arranged at intervals, a deformation gap is reserved between every two adjacent engaging arms, and second engaging convex portions interacting with the first engaging convex portion are provided at outer sides of the engaging arms.

32. The safety syringe according to claim 31, wherein a second guide surface is provided on an outer side of a distal end of each of the engaging arms.

33. The safety syringe according to claim 23, wherein a sealing ring is arranged between the distal end of the retractable rod and the retraction cavity.

34. The safety syringe according to claim 23, wherein an inwardly inclined sloping surface is provided on a distal end side of each of the guide blocks.

35. The safety syringe according to claim 23, wherein a plurality of cantilevers are arranged at the proximal end of the retractable rod, the guide blocks are arranged at free ends of the cantilevers, and guide sloping surfaces are provided on proximal end sides of the guide blocks.

36. The safety syringe according to claim 34, wherein a plurality of cantilevers are arranged at the proximal end of the retractable rod, the guide blocks are arranged at free ends of the cantilevers, and guide sloping surfaces are provided on proximal end sides of the guide blocks.

37. The safety syringe according to claim 23, wherein the needle holder comprises a holder body connected to the syringe barrel and a holder body II detachably connected to a base, and the injection needle is fixedly connected to the holder body II.

38. The safety syringe according to claim 37, wherein a circumferential limiting structure is provided between the holder body I and the needle holder mounting portion.

39. A safety syringe, comprising at least:
a syringe barrel, the syringe barrel being provided with an inner cavity extending from a proximal end to a distal end, a needle holder mounting portion being provided at the distal end of the inner cavity;
a needle holder, an injection needle being arranged at a distal end of the needle holder, a first engaging portion being provided at a proximal end of the needle holder, and the needle holder entering from the proximal end of the inner cavity and being mounted on the needle holder mounting portion;
a ring plug, an annular boss being provided at the proximal end of the needle holder, the ring plug being mounted between the annular boss and an inner wall of the inner cavity in an interference manner, an annular cavity for accommodating the ring plug being formed between the annular boss and the distal end of the inner cavity, and the ring plug being in clearance fit with the needle holder and the inner wall of the inner cavity in the annular cavity;
further comprising:
a push rod, the push rod being located inside the inner cavity, a retraction cavity being provided inside the push rod, a piston being mounted at a distal end of the push rod, a retractable rod coupling chamber being arranged at a distal end of the retraction cavity, a maximum aperture of the retractable rod coupling chamber being smaller than an aperture of the retraction cavity, and a first stopper portion being provided inside the retractable rod coupling chamber;
a retractable rod, comprising a retractable rod body that is in interference fit with the retractable rod coupling chamber, wherein a second stopper portion for interacting with the first stopper portion is provided at a proximal end side of the retractable rod body, a second engaging portion for adapting to the first engaging portion is provided at an end portion of a distal end of the retractable rod body, and a first spring mounting portion is provided at a proximal end of the retractable rod body; and
a tension spring, with one end being mounted on the first spring mounting portion, and the other end being fixedly connected to an end portion of a proximal end of the retraction cavity.

40. The safety syringe according to claim 39, wherein a transition conical surface is arranged between the retraction cavity and the retractable rod coupling chamber, and a sealing element for abutting against and sealing with the transition conical surface is arranged on the retractable rod.

41. The safety syringe according to claim 39, wherein the retractable rod body is conical, and an outer diameter of the retractable rod body gradually increases from the distal end to the proximal end.

42. The safety syringe according to claim 39, wherein the inner wall of the inner cavity is provided with a first protrusion in a circumferential direction at a position corresponding to a proximal end side of the annular boss for preventing the ring plug from moving towards a proximal end.

43. The safety syringe according to claim 39, wherein the first engaging portion comprises an engaging groove extending from an end face of the proximal end of the needle holder to the distal end, and a first engaging convex portion is provided on an inner wall of the engaging groove.

44. The safety syringe according to claim 43, wherein a first guide surface is provided at an entrance of the engaging groove.

45. The safety syringe according to claim 43, wherein the second engaging portion comprises a clamping groove for accommodating the first engaging convex portion, and a clamping groove wall for limiting the first engaging convex portion within the clamping groove is provided at a distal end side of the clamping groove.

46. The safety syringe according to claim 45, wherein a conical guide portion is provided on a side of the clamping groove away from the retractable rod body.

47. The safety syringe according to claim 44, wherein the second engaging portion comprises a clamping groove for accommodating the first engaging convex portion, and a clamping groove wall for limiting the first engaging convex portion within the clamping groove is provided at a distal end side of the clamping groove.

48. The safety syringe according to claim 47, wherein a conical guide portion is provided on a side of the clamping groove away from the retractable rod body.

49. The safety syringe according to claim 39, wherein a proximal end of the push rod is provided with a gland, the gland is provided with a second spring mounting portion, and a proximal end of the tension spring is connected to the second spring mounting portion.

50. The safety syringe according to claim 39, wherein the second stopper portion is provided with a guide surface.

51. The safety syringe according to claim 40, wherein the second stopper portion is provided with a guide surface.

52. The safety syringe according to claim 41, wherein the second stopper portion is provided with a guide surface.

53. The safety syringe according to claim 42, wherein the second stopper portion is provided with a guide surface.

54. The safety syringe according to claim 43, wherein the second stopper portion is provided with a guide surface.

55. The safety syringe according to claim 44, wherein the second stopper portion is provided with a guide surface.

56. The safety syringe according to claim 46, wherein the second stopper portion is provided with a guide surface.

57. The safety syringe according to claim 48, wherein the second stopper portion is provided with a guide surface.

58. The safety syringe according to claim 49, wherein the second stopper portion is provided with a guide surface.
